(19) United States
(12) Patent Application Publication
(10) Pub. No.: US 2008/0187612 A1
(43) Pub. Date: Aug. 7, 2008

(54) Delivery Systems

(76) Inventors: David Kannar, Victoria (AU); Barry James Kitchen, Victoria (AU); Simon John Armstrong, Victoria (AU)

Correspondence Address: REED SMITH LLP, P.O. BOX 488, PITTSBURGH, PA, 15230-0488, US

(21) Appl. No.: 11/915,839

(22) PCT Filed: Jun. 2, 2006
(86) PCT No.: PCT/AU2006/000761

§ 371 Date: Feb. 27, 2008

(30) Foreign Application Priority Data
Jun. 3, 2005 (AU) 2005902906

(51) Int. Cl. A61K 36/18; A23G 1/36; A61K 47/46

(52) U.S. Cl. 424/776; 426/631; 426/519; 426/321; 426/281; 426/93; 514/783

(57) ABSTRACT

There is provided drug delivery systems for bioactive substances. In particular, there is provided a chocolate product containing bioactive compounds as well as desirable flavours and deliverability.

### FIELD OF THE INVENTION

The invention relates to delivery systems for bioactive substances, including a method for delivering bioactive powders which are difficult to disperse evenly in conventional vehicles and a method for delivering liquids (including aqueous liquids) in a fat based delivery system.

The invention also provides a chocolate product having a desired flavour and organoleptic properties as well as an evenly distributed bioactive substance content.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Identification of a formulation that affords clinically adequate blood levels after oral administration is a major milestone in the development and commercialisation of a drug molecule.

The oral route is the most popular and convenient route of drug administration for those drugs which can survive the acid of the stomach, which are resistant to enzymatic attack and which are absorbed across gastrointestinal (GI) membranes. When a drug is administered orally, a complex cascade of events must occur before the drug is absorbed into the bloodstream and available to exert the desired pharmacological effect. Of key importance are the chemical characteristics of the drug and the nature and composition of the formulation as these play crucial roles in defining the release from the delivery system, dissolution within the gastrointestinal tract lumen and permeation across the intestinal mucosa.

The functions of the GI tract are the digestion and absorption of food and other nutrients. The natural processes in the GI tract frequently influence the absorption of drugs. The pH of the GI tract contents and the presence of enzymes, foodstuffs, bile salts, fat and microbial flora will all influence drug absorption. While most drugs are absorbed by passive diffusion across the lipid membranes separating the GI tract contents from the rest of the body, certain molecules that resemble naturally occurring substances are actively transported by special mechanisms.

The stomach is not the principal area of absorption in the GI tract and the main site of absorption is the small intestine. The stomach is still important in relation to drug absorption because it changes volume dramatically during a day and may contain an amount of fluid which will vary in volume between a few millilitres and over a litre with varying concentrations of hydrochloric acid and gastric juice. The volume of the gastric contents will determine the concentration of a drug in the stomach. The time the drug or dosage form resides in the stomach will determine many aspects of absorption. If the drug is absorbed lower down the GI tract, the rate of travel of the drug will determine the delay before absorption begins. If the drug is sensitive to the environment in the stomach then the degree of sensitivity will determine the amount of degradation before absorption. The stomach empties liquids faster than it does solids. The rate of transfer of gastric contents to the small intestine is retarded by the activity of receptors sensitive to acid, fat, osmotic pressure and amino acids in the intestine and stimulated by material arriving from the stomach. Acids and fats slow down gastric emptying. The nature of the dose form, whether solid or liquid, whether acid or alkaline, whether aqueous or oily, may thus influence gastric emptying.

The gastrointestinal tract presents a pharmaceutics conundrum to a formulator because of the competing physiochemical and clinical pharmaceutical issues required for effective absorption. The general rule is that only drugs which are in a liquid form are absorbed from the GI tract. The choice of formulation is therefore crucial to oral drug action and efficacy. For example, absorption of water insoluble, lipophilic drugs following oral administration is generally low and highly variable due to their low solubility in the aqueous environment of the gastrointestinal tract.

Lipophilic drugs, compounds with a high solubility constant and acidic drugs may also penetrate the gastrointestinal lipid membrane causing irritation and therefore must be taken with food to reduce side effects such as nausea.

The variation in bioavailability demonstrated by lipophilic drugs and nutrients has limited their use in medicine, as the required dosage is unpredictable. If a dose could be predictably provided to humans and animals, utility of these lipophilic nutrients and drugs would be greatly increased.

It is important for a drug to be evenly dispersed in a formulation so that during manufacture each unit has the same dose of the drug. Some powders are difficult to disperse evenly because they form small “balls” when in contact with a liquid. These balls are very difficult to break up during mixing and the resultant formulation has a patchy dispersion of the drug. It is possible to overcome this problem in some instances by using other excipients such as emulsifiers to assist with dispersion.

Cocoa Butter and Chocolate

Fats derived from a variety of plant sources are widely used in food and confectionery applications. Among these are cocoa butter, palm and palm kernel oil, coconut oil, peanut oil, soybean oil, cottonseed oil, sunflower oil and illipe butter and shea butter. Each fat is characterised by a different triglyceride make up, both in the percentage of the individual fatty acids present and their combination and position on the glycerol backbone of the triglyceride. This in turn results in different melting profiles and percentages of solid to liquid fat present at different temperatures. These fats provide the necessary functional (principally textural) properties to the foodstuffs and depending upon their melting characteristics deliver important mouth feel and organoleptic effects (eg cooling, waxiness).

Fats which have a sharp melting curve in the range 30-37° C. and which melt at around body temperature (35-37° C.) provide desirable organoleptic properties. This desirable characteristic is found in cocoa butter and provides the exceptional mouth feel and organoleptic properties of chocolate products. The different fractions of cocoa butter have different melting points. However, cocoa butter as a whole typically has between 60-70% of its fat in a solid form at 25° C. and as the temperature increases to 32-33° C. the percentage of solid fat present falls to less than 10%. By 37-38° C. this figure is close to zero (Kattenberg, H. R., Manufacturing Confectioner, January 1981).

Cocoa butter is a multicomponent mixture of triglycerides and trace components. Approximately 85% of cocoa butter consists of just three triglycerides: POP, POS and SOS where the letters refer to the fatty acids attached to the glycerol base (P is palmitic, S is stearic and O is oleic). The exact composition depends on factors such as growing conditions.

Cocoa butter also has very complex crystallisation properties in which it crystallises into several different forms as the liquid fat is cooled. There are four main polymorphic crystal forms.
gamma form, melting point 17° C., unstable
alpha form, melting point 21-22° C., unstable
beta prime form, melting point 27-29° C., unstable
beta form, melting point 34-35° C., stable

The objective during the manufacture of chocolate is therefore to ensure that the cocoa butter in the matrix is in the most stable beta form prior to cooling to solidify the product. Failure to control this results in poor colour, fat instability and poor textural properties of the final product.

Cocoa butter can be combined with (for example) milk or butter fat, which further changes the melting profile and percent of solid or liquid fat present at any particular temperature. Milk fat and butter fat are two of the few fats that are compatible with cocoa butter. For example, the percentage of liquid fat present in a cocoa butter to milk fat blend of 82/28 can reach 75% at 30° C., compared to only around 35% at 30° C. for pure cocoa butter. At 20° C. pure cocoa butter only has around 15% liquid fat present while the cocoa butter milk fat blend is around double this value (approximately 30%) (Chocolate, Cocoa and Confectionery—Science and Technology by Bernard W. Minifie, 1999 4^{th} Ed, page 85-110, Aspen Publications).

Cocoa butter's melting profile at physiological temperatures has led to its use as a base for suppositories.

Cocoa butter equivalents (CBE's) are vegetable fats derived from palm and shea oils, illipe (borneo tallow or tengkawang), sal, shea, kokum gurgi, and mango kernel. CBE's are chemically and physically very close to cocoa butter and can provide advantages such as improved heat resistance when compared with cocoa butter.

Some fats (eg palm oil) are modified chemically (hydrogenation, interesterification) in order to change the melting characteristics which mimic the unique melting properties of cocoa butter. However, these modified fats never quite deliver on the melting and thus the desired organoleptic properties. Such modified fats are more waxy and thus provide a lower organoleptic quality than pure cocoa butter.

Flavoured Chocolate

The addition of drugs to chocolate can introduce unpleasant flavours to chocolate, eg unwanted bitterness. Therefore it is often necessary to add flavours to the chocolate to mask the powder flavour.

However, the addition of flavours to chocolate can present a challenge because the addition of aqueous compounds can disrupt water activity and shelf life, while the addition of lipophilic compounds can interfere with tempering, setting and organoleptic properties of chocolate. Wine and flavours are difficult to incorporate into chocolate because it is compatible with even small amounts of aqueous material. Amounts of only 1-2% of water will change the flow of chocolate so that its ability to be moulded into shapes, texture, mouth feel and palatability are reduced.

Some attempted commercial chocolate products containing polyphenols have failed to attract sales because of the unpleasant flavour. There is thus a need for a method to flavour chocolate which provides a real flavour experience, especially wine flavoured chocolate.

Liquid Drugs and Chocolate

The incompatibility of chocolate with many liquids has also made it difficult to use chocolate to deliver drugs which are a liquid. There is thus a need for a delivery system which enables the delivery of liquid drugs in a chocolate based system.

A drug delivery method-that will enable the even dispersion of powders is also needed. Further, a drug delivery system that achieved this and also improved dissolution profile, increased solubility or did not reduce absorption or bioavailability would offer significant advantage.

### SUMMARY OF THE INVENTION

It has been surprisingly found that bioactive powders, which have previously been difficult to handle because they typically “ball up” or require emulsifiers, are easily and evenly dispersed using a drug delivery system comprising cocoa butter or similar lipids compatible with chocolate (eg cocoa butter substitutes, cocoa butter equivalents, cocoa butter replacers). The combination of the bioactive powder and cocoa butter, or equivalent lipid, may have the advantages of improving physiochemical properties including dissolution, solubility and pharmacokinetic parameters such as delivery, transport, absorption, bioavailability and efficacy of the bioactive ingredient. It may also limit erosive damage to the stomach lining or reducing proteolysis, hydrolysis or similar action causing breakdown of the lipophilic bioactive compound.

Further, it has been found that bioactive substances can be incorporated into chocolate products using inclusions, which is particularly useful for delivery of liquid drugs in chocolate products.

In addition, it is now possible to produce a chocolate product with an improved wine flavour experience and which contains an even distribution of phenolic powders.

According to the first aspect of the invention, there is provided a drug delivery system for bioactive powders comprising the bioactive powder and a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, wherein the bioactive powder is substantially evenly distributed throughout the delivery system.

In a preferred embodiment, the lipid is cocoa butter, a cocoa butter equivalent or a cocoa butter fraction (such as stearic acid) so that the drug delivery system can be more easily incorporated into cocoa products such as chocolate.

The first aspect of the invention also includes a method for delivering bioactive powders, the method comprising the step of combining the bioactive powder with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, wherein the bioactive powder is substantially evenly distributed throughout the lipid.

The first aspect of the invention further includes the use of a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof in the manufacture of a formulation comprising a bioactive powder, wherein the bioactive powder is substantially evenly distributed throughout the formulation.

Where used herein the term “sharp melting curve in the range from 30 to 37° C.” refers to the lipid having a melting curve similar to that of chocolate or cocoa butter in this temperature range.

Where used herein the term “substantially evenly distributed” refers to distribution of the bioactive powder in as consistent a manner as possible, so that if any part of the composition is tested the content of bioactive powder will be similar to that of another part within the range of tolerance usually allowed for therapeutic products. At this time there is no specific standard for chocolate products, however there are standard discrete tolerances of deviation for therapeutic products, such as tablets.

A person skilled in the art will know which lipids are miscible with chocolate and thus can be used in the invention, such as milk fat or butter fat. These lipids include lipids which do not interfere with the crystalline structure of cocoa butter in chocolate. These lipids also include those lipids which can be used in specific quantities without disturbing the crystalline structure, even though at higher quantities such lipids might be incompatible. Lipids which cannot be used in the invention are those lipids which are incompatible with chocolate, that is, will disturb the crystalline structure and will cause ‘bloom’ (white coating).

In one embodiment, milk fat can be used to assist with dispersing a bioactive powder in cocoa butter to achieve the desired dose. Milk fat is a lipid which is compatible with cocoa butter and can be mixed in the cocoa butter to provide more liquid fat at normal product storage temperatures (18-20° C.). Such a milk fat/cocoa butter mixture will retain the ability to rapidly change its liquid to solid profile at body temperatures in the mouth and gut.

A person skilled in the art will realise that the invention is useful for the administration of lipophilic bioactive powders which have a highly temperature dependent solubility over the temperature range of approximately 4 to 37° C. or a high entropy of solution in lipid-based formulations. Examples of such compounds include, but are not limited to, CoQ10, vitamin A and its derivatives, vitamin E and its derivatives and vitamin K and its derivatives. An important feature of the drug delivery system of the present invention is the ability of crystallised or precipitated lipophilic bioactive powders present in the formulation to rapidly re-dissolve following exposure to physiological temperature (37° C.) for at least about 10 minutes. This ensures that the lipophilic bioactive powder is solubilized which improves absorption of the lipophilic bioactive powder by humans or animals having a physiological temperature of about 37° C. The present invention thus removes the need for redissolution of any precipitated drug which is an issue with standard soft gel capsules. It is also expected that the present invention will enable increased and/or less variable bioavailability. For example, some drugs, such as mebendazole, have improved bioavailability when administered with fatty foods.

Of course, hydrophilic bioactive powders would be dispersed in the delivery system of the present invention but not dissolved. These hydrophilic bioactive powders would dissolve into the aqueous environment of the stomach after administration.

The first aspect of the invention also provides a cocoa product comprising a bioactive powder, wherein the bioactive powder is dispersed in a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, wherein the bioactive powder is substantially evenly dispersed throughout the cocoa product.

Preferably the cocoa product is chocolate.

The high fat and low moisture environment of chocolate may require that certain bioactive powders be combined with the selected lipid prior to adding the lipid to the rest of the chocolate ingredients. This ensures that the lipophilic bioactive compound is properly solubilized and substantially evenly distributed.

The first aspect of the invention also provides for a method for manufacturing a chocolate product comprising a bioactive powder, said method comprising:
first, combining the bioactive powder with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the powder is substantially evenly distributed in the lipid, and then
combining the lipid mixture with the other chocolate ingredients until the lipid mixture is substantially evenly dispersed in the chocolate product.

The polyphenols content of the chocolate product can be increased by adding low glycaemic index sugars and high phenolic sugars as part of the sugars in the chocolate ingredients. Examples of such sugars are disclosed in international patent application no 2005/117608.

A person skilled in the art will realise that some bioactive powders will be heat sensitive and therefore it will be necessary to pay careful attention to the chocolate manufacturing process to ensure that the temperature of the mixture is not too high. For example, in the manufacture of some dark chocolates, temperatures may exceed 60° C. as a result of mechanical energy input.

Where used herein the term “bioactive powder” refers to all substances having a biological effect which are available in powdered form and includes, but is not limited to, pharmaceuticals, nutrients, nutriceuticals, herbal extracts, vitamins, phytochemicals and health supplements. Such bioactive powders include but are not limited to grape skin polyphenols, aspirin and poorly absorbed pharmaceutical compounds such as ubiquinol, retinol, tocopherol, mebendazole and cefaclor monohydrate. Preferably the bioactive powder is a polyphenol sourced from cocoa, sugar cane, sugar beet, grapes, wine and mixtures thereof.

The compounds in the bioactive powder may be any suitable form including but not limited to its free form, salts, analogues, derivatives, prodrugs, hydrated forms and complexes. In addition, the bioactive powder may interact with the selected lipid of the present invention. Examples of such interaction may be complexation, covalent bonding and hydrogen bonding. This interaction may lead to improved dissolution, solubility and pharmacokinetic parameters including delivery, transport, absorption, bioavailability and efficacy.

The first aspect of the invention also provides a method for improving the bioavailability of lipophilic bioactive powders, said method comprising the step of combining the lipophilic bioactive powder with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the bioactive powder is substantially evenly dispersed throughout the lipid.

The first aspect of the invention also provides a method for minimising the level of degradation of a bioactive powder, said method comprising the step of administering the bioactive powder in combination with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the bioactive powder is substantially evenly dispersed throughout the lipid.

The first aspect of the invention also provides a method for minimising the interaction between a bioactive substance and the stomach lining, said method comprising the step of administering the bioactive powder in combination with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, wherein the bioactive powder is substantially evenly dispersed throughout the lipid.

According to a second aspect of the invention, there is provided a method for incorporating bioactive substances, such as polyphenols, into cocoa products, said method comprising the steps of:
combining the bioactive substance with an infusion liquid,
infusing the bioactive liquid mixture into inclusions; and then
combining the infused inclusions with a cocoa product.

The second aspect of the invention also provides a method for improving the bioavailability of bioactive substances, said method comprising the steps of:
combining the bioactive substance with an infusion liquid,
infusing the bioactive liquid mixture into inclusions; and then
combining the infused inclusions with a cocoa product.

The second aspect of the invention also provides a method for minimising the level of degradation of a bioactive substance, said method comprising the steps of:
combining the bioactive substance with an infusion liquid,
infusing the bioactive liquid mixture into inclusions; and then
combining the infused inclusions with a cocoa product.

The second aspect of the invention also provides a method for minimising the interaction between a bioactive substance and the stomach lining, said method comprising the steps of:
combining the bioactive substance with an infusion liquid,
infusing the bioactive liquid mixture into inclusions; and then
combining the infused inclusions with a cocoa product.

The infusion liquid can be any appropriate solvent for the bioactive substance which will be absorbed into the inclusion. Typical examples include alcohols, non-alcoholic beverages, and grape skin extract. Preferably, the liquid is grape skin extract which is an alcohol based liquid having low water activity and pH. It is also thought that grape skin extract does not adversely affect the plate count. Drug solutions or drug complexes may be further included into the infusion mixture. A range of natural and synthetic compounds can also be used as the infusion mixture without the need for further a solvent.

Preferably, the infusion liquid further comprises flavours.

The flavour will depend on the specific note and profile desired to be highlighted in the finished product. The flavour may have an alcoholic, monosaccharide, polysaccharide, polydextrose, polydextrin, dextrin, polyol, starch, propylene glycol, vegetable oil, triglyceride or other suitable base/carrier. The base/carrier is selected on the basis of suitability to the bioactive compound being delivered and may also include suitable antioxidants, food acids, humectants or preservatives as required.

A non-alcoholic infusion mix can be used by substituting the wine by a non-alcoholic/de-alcoholic liquid. In addition, a range of non-alcoholic or de-alcoholised flavours can be added to the infusion mix to improve taste and deliverability of the inclusions in the chocolate. The flavour of the inclusions can enhance the flavour of the whole chocolate product.

Bioavailability of the infusion mix and bioactive compounds may be enhanced by a variety of compounds including but not limited to surfactants, solubilisers, organic acids and emulsifiers known in the pharmaceutical food chemistry art and selected depending on the physiochemical properties of the bioactive compound. Uptake of the infusion mixture by the inclusions can also be improved using a variety of compounds such as surfactants and solubilisers.

The inclusion can be any appropriate absorptive substance. Typical examples include dried fruit (currants, raisins, cranberries, sultanas, sun muscats), freeze dried fruit, dried fruit pastes, fruit purees, extruded fruit particles, puffed grains, baked grain inclusions, processed cereals and cereal fractions (such as bran), and synthetic substitutes (such as resistant starches, extended release pharmaceutical matrices eg. durettes, durilles, and microcrystalline cellulose coated microparticles). In many cases, the size of the inclusion will need to correspond to the size of the manufacturing equipment, eg depositor and depositing head.

In a preferred embodiment, the inclusions are dried prior to adding to a cocoa product to avoid any of the infusion liquid disturbing the cocoa product structure. The inclusions can be dried using any known methods or temperatures suitable for the properties of the bioactive compound. Examples include freeze drying, heat pump air convection, conduction, vacuum drying and sun drying. The drying temperature, method and conditions will vary depending on the bioactive compound. For example, heat sensitive bioactive compounds may be dried at a lower temperature.

Where used herein the term “bioactive substance” refers to all substances having a biological effect includes, but is not limited to, pharmaceuticals, nutrients, nutriceuticals, herbal extracts, vitamins, phytochemicals and health supplements. Such bioactive substances include but are not limited to grape skin polyphenols, aspirin and poorly absorbed pharmaceutical compounds such as ubiquinol, retinol, tocopherol, mebendazole and cefaclor monohydrate.

Preferably the bioactive substance is a polyphenol sourced from a range of sources including cocoa, sugar cane, sugar beet, molasses, grapes, wine, fruit (berries, drupes, pomes, tropical fruits, juices), vegetables (bulbs, roots, tubers, leaves, stems), herbs, spices, beans, pulses, grains (barley, buckwheat, corn, millets, oats, rice, rye, sorghum, wheat), nuts (almonds, betel nuts, cashews, hazelnuts, peanuts, pecans, walnuts), oilseeds, plant oils, tea, coffee, beer, cider, seeds and mixtures thereof.

The polyphenols content of the infusion liquid, and thus the inclusions, can be increased by adding low glycaemic index sugars and high phenolic sugars to the infusion liquid. Examples of such sugars are disclosed in international patent application no 2005/117608.

The compounds in the bioactive substance may be any suitable form including but not limited to its free form, salts, analogues, derivatives, prodrugs, hydrated forms and complexes. In addition, the bioactive substance may interact with the selected infusion liquid of the present invention. Examples of such interaction may be complexation, covalent bonding and hydrogen bonding. This interaction may lead to improved dissolution, solubility and pharmacokinetic parameters including delivery, transport, absorption, bioavailability and efficacy.

The inclusions can be infused with the infusion mixture using any methodology. Typically, the inclusions are mixed with the infusion mixture in a vessel with regular mixing during the infusion period. Another method which can be used is cryovac.

This method avoids the difficulties with incorporating liquids into the chocolate mixture and minimises the effect of the liquids on the resultant quality of the chocolate.

The inclusions also provide an advantage in enabling a burst of flavour on the tongue when consumed which enhances the overall flavour experience when consuming the cocoa product. This is particularly advantageous with respect to the wine flavours since real wine is infused into the inclusions.

According to a third aspect of the invention there is provided a wine chocolate product comprising:
(a) wine flavoured chocolate comprising chocolate, flavours, and grape polyphenols dispersed in a lipid selected from the group consisting of cocoa butter, cocoa butter equivalents, cocoa butter substitutes, other lipids having a sharp melting curve in the range from 30 to 37° C. and which melt in the range from 35 to 37° C., other lipids which are miscible with chocolate and mixtures thereof, wherein the bioactive powder is substantially evenly distributed throughout the wine flavoured chocolate; and
(b) wine flavoured inclusions comprising inclusions which have been infused with a liquid mixture comprising wine, grape polyphenols and flavours.

The wine chocolate product can be in the form of block chocolate or chocolate coated inclusions. The wine chocolate product may also be in the form of chocolate centred filled with the inclusions and wine flavoured filling moulded into blocks wherein the pips each have about the same polyphenol content. The inclusions may also be included into another food product (such as a biscuit or snack bar) which is then coated in the chocolate.

The third aspect of the invention also provides a method of enhancing the absorption of a polyphenol into the blood of a human comprising administering to the human a wine chocolate product according to the invention.

The third aspect of the invention also provides a method of promoting vascular health of a human comprising administering to the human a wine chocolate product according to the invention.

### EXAMPLES

Various embodiments/aspects of the invention will now be described with reference to the following non-limiting examples.

### Example 1

This example demonstrates the use of the invention to produce a commercial chocolate product containing polyphenols as the bioactive powder.

Infusion of Currants

Infusion Mixture: The following mixture (25 litres) was sufficient to infuse 25 batches of currants (each batch 5 kg)
20 litres Wine (red wines such as shiraz, merlot, pinot noir, fortified wine such as port, Muscat, marsala or botrytis, white wines such as chardonnay, sauvignon blanc, riesling, and sparkling wines)
5 litres Vinlife™ (Tarac Technologies) Grape Skin Extract (liquid)
125 ml Flavouring

Mix the above well in a large plastic vessel at room temperature and ensure that the grape skin extract and flavour is well mixed with the wine.

Infusing the Currants: Weigh 5 kg of small dried currants into each of 25 (15-20 1 size) plastic tubs (with lids) and then add 1 litre of the Infusion Mixture. Mix well with a metal or wooden stirrer and ensure all of the currants are coated with the mixture. Place lid on tub and mix again by gentle swirling and shaking. Allow each sealed tub to stand at room temperature for 30 minutes and mix again by swirling and shaking. Repeat again 30 minutes later then store the tubs in a cool room (±5° C.) overnight. Remove the tubs as required the next morning and mix each one in turn, stand for 30 minutes and mix again.

Filter each tub when required for addition to the flavoured and tempered chocolate and discard filtrate. Shake the infused currants well in the filter to remove as much surface liquid as possible. Spread the infused currants on a drying rack and place in a warm room (40° C.) with air flowing across the currants overnight.

Preparation of Chocolate Containing Grape Seed Powder and Flavouring

Base Chocolate Recipe (per 500 kg (0.5 t batch)):

| Ingredient | Amount |
|---|---|
| Castor Sugar | 200 kg |
| Full cream milk powder | 70 kg |
| Cocoa Liquor (Ivory Coast) | 175 kg |
| Cocoa Butter Deodorised | 50 kg |
| Soy Lecithin - (Add half initially and half | 2.5 kg |
| 30-60 mins before finishing conching cycle) | |
| PGPR - (Add half, 1 kg, initially and | 2.0 kg |
| remainder after addition of flavours (to | |
| reduce viscosity).) | |
| Natural Vanilla Flavou r- (Add 30 mins | 2.0 kg |
| before finishing conching cycle) | |

Add to conche in correct sequence and conche for 12-16 hours at 40° C. until average particle size of chocolate reaches less than 20μ (range 18μ-20μ). The chocolate has a milk fat to cocoa butter ratio of 0.13.

Preparation Of Seed Powder (for 0.5 tonne batch of chocolate): Weigh out 2.25 kg of Vinlife™ (Tarac Technologies) Grape Seed Powder and add to 5 kg of melted (45° C.) cocoa butter. Add slowly with stirring and ensure the powder is dispersed substantially evenly throughout the cocoa butter. Avoid incorporation of air whilst mixing, but ensure that the powder is well dispersed in the cocoa butter.

Addition Of Seed Powder To Chocolate: Add the cocoa butter/grape seed powder mixture to the conche in the last hour of the conching cycle. Use a further 5 kg of melted cocoa butter to rinse the remnant of the cocoa butter/grape seed powder mixture from the vessel.

The chocolate was then flavoured as a wine variety. The real varietal wine flavour in the chocolate can be enhanced by adding a range of flavours that not only enhance the flavour but serve to reduce bitterness when higher than usual amounts of polyphenols are added to promote health. A person skilled in the art of flavour chemistry will know which mix of flavours may be used to improve palatability, mouth feel and other organoleptic properties.

Addition Of Infused Currants To Chocolate

The filtered and drained currants (approx 5.0-5.5 kg) were mixed with 40 kg of the flavoured and tempered chocolate. The mixture must be mixed well to ensure an even distribution of the currants.

The currant/chocolate mixture is then moulded and cooled.

By using dried currants or fruit infused with wine and water-soluble polyphenols dispersed in cocoa butter, difficulties typically experienced with addition to foods such as chocolate can be overcome. Taste can further be improved using wine flavours and a uniquely palatable product can be produced with enhanced polyphenol content, antioxidant and ACE inhibitory activity.

Incorporation of both wine and water soluble polyphenol extract with a carrier such as dried fruit using the conditions described, preserves the infused product despite increased water activity and moisture content. In addition it results in a product with superior organoleptic qualities and stable shelf life at 25° C.

### Example 2

This example investigated the antioxidant levels in phenolic-fortified milk chocolate.

Method

The antioxidant capacity of 6 pieces of control milk chocolate (1 piece from each row of an approximately 100 g block) and 12 pieces of phenolic-fortified milk chocolate (2 pieces from each row alternating 1^{st} and 3^{rd}, 2^{nd} and 4^{th}) were chosen for assay. The chocolate was provided by Cool Health Pty Ltd. The chocolate was prepared as per Example 1. A sample of each, weighing between 1.7 and 2 g, was weighed accurately and added to a 50 ml tube. The chocolate samples were defatted by the addition of 20 ml heptane. The samples were centrifuged and the heptane decanted. The samples were left open in a fume hood to remove traces of heptane. The antioxidants were extracted using 2×20 ml aliquots of 80% methanol, the first a 2 hour extraction and the second an overnight extraction. The primary and secondary extract were added together and assayed in duplicate using the ABTS method after a 5-fold dilution in water.

Results

| Control milk chocolate | | Fortified milk chocolate | |
|---|---|---|---|
| Sample | Antioxidant Capacity | Sample | Antioxidant Capacity |
| (Row, | (mg catechin | (Row, | (mg catechin |
| Position) | equivs/g) | Position) | equivs/g) |
| 1, 1 | 1.638 | 1, 2 | 1.832 |
| 2, 1 | 1.578 | 1, 4 | 1.857 |
| 3, 4 | 1.572 | 2, 1 | 2.022 |
| 4, 2 | 1.634 | 2, 3 | 1.859 |
| 5, 3 | 1.547 | 3, 2 | 2.370* |
| 6, 4 | 1.557 | 3, 4 | 1.924 |
| | | 4, 1 | 1.914 |
| | | 4, 3 | 1.937 |
| | | 5, 2 | 1.971 |
| | | 5, 4 | 1.936 |
| | | 6, 1 | 2.016 |
| | | 6, 3 | 1.900 |

Discussion

The antioxidant capacity of the control chocolate was 1.587±0.039 mg catechin equivalents per gram (mean±standard deviation). The antioxidant capacity of the phenolic-fortified chocolate was 1.961±0.142 mg catechin equivalents per gram. One of the phenolic-fortified samples (piece 3,2) was appreciably higher than the others. If this piece was restricted from analysis the result for the remaining 11 samples was 1.924±0.062 mg catechin equivalents per gram. This represents an increase of 21.2% compared with the control chocolate.

### Example 3

This example investigates the dispersion of red wine polyphenols into cocoa butter.

Grape seed powder (purchased from Vinlife™, Tarac Technologies, Australia) is difficult to disperse in an aqueous environment and this is a common phenomenon shared with many powders containing a complex mixture of lipophilic and hydrophilic materials. “Balling” and “fisheying” can occur, making the powders difficult to evenly disperse in the delivery material which then would not disperse evenly through a product such as chocolate. Grape seed powder contains many polyphenols which have a beneficial therapeutic effect.

Cocoa butter was melted and held at 40-45° C. To this was added the grape seed powder at a rate of 400 mg per ml of cocoa butter. With gentle agitation, the powder immediately dispersed and “solubilized” into the cocoa butter. The cocoa butter mixture (10 ml) was then added to 1 kg of a typical dark chocolate formulation and mixed by gentle agitation. This theoretically delivered 400 mg of the bioactive powder to each 100 g of chocolate. The chocolate was then moulded and cooled.

The product was assessed for its organoleptic properties with a particular emphasis on any effect of the powder particles on the mouth feel of the product. The powder particles were found to have no effect on the organoleptic properties or the mouth feel.

The chocolate (100 g) was divided into 10 equal portions and each was analysed for the level of grape seed powder present. The results showed that the mean (range) values for the level of bioactive powder present in the samples was 38.5 (35.6-42.8) mg per 10 g thus indicating an even dispersion of the bioactive material throughout the matrix.

### Example 4

This example investigates the inclusion of red wine polyphenols into dried currants.

Method

A liquid infusion mix consisting of equal amounts of regular shiraz red wine (approximately 12.5% weight for weight alcohol) and a commercial grape skin extract (Vinlife™, Tarac Technologies, Australia) high in antioxidants and polyphenols were supplemented with a grape seed powder (Vinlife™) at the rate of 20 mg of powder per ml of the infusion mix.

This infusion mix (20-25 ml) was added to 100 g of dried currants and allowed to passively infuse with gentle mixing at 20° C. for a period of 24 hours. Sampling was carried out at regular intervals to determine the rate of infusion into the currants. Typically, half of the infusion mix had infused into the currants within 4-5 hours and by 24 hours around 90-95% of the infusion mix had been taken up by the currants.

When the filtered and partially dried infused currants were then incorporated into a typical chocolate matrix (either dark or milk chocolate formulations) there was no detrimental effect on the properties of the chocolate from a textural or handling properties point of view.

Furthermore, the taste and keeping quality of the chocolate containing the antioxidant infused currants was excellent.

The currants were analysed and the results are in the following table.

| Analysis | Un-infused currants | Infused currants |
|---|---|---|
| Moisture (g/100 g) | 14.6 | 26.6 |
| Water activity | 0.54 @ 24° C. | 0.75 @24° C. |
| Alcohol content | NA | 2.14% |
| Standard plate count per g | | |
| Initial | 1,700 | 900 |
| 1 week, 21° C. | 600 | 2,100 |
| 2 weeks, 21° C. | 800 | 400 |
| 3 weeks, 21° C. | 1,200 | 600 |
| 6 weeks, 21° C. | 500 | 1,400 |
| Yeasts per g | | |
| Initial | <100 | <100 |
| 1 week, 21° C. | <100 | <100 |
| 2 weeks, 21° C. | <100 | <100 |
| 3 weeks, 21° C. | <100 | <100 |
| 6 weeks, 21° C. | <100 | <100 |
| Moulds per g | | |
| Initial | <100 | <100 |
| 1 week, 21° C. | <100 | <100 |
| 2 weeks, 21° C. | <100 | <100 |
| 3 weeks, 21° C. | <100 | <100 |
| 6 weeks, 21° C. | <100 | <100 |
| pH | | |
| 3 weeks, 21° C. | 4.0 | 3.9 |
| 6 weeks, 21° C. | 3.8 | 3.9 |

### Example 5

This example illustrates an alternative infusion liquid preparation.

The following mixture (25 litres) was sufficient to infuse about 125 kg of currants (inclusions). The ratio of the infusion mixture components may be varied according to the wine flavour being used. The amount of infusion mixture to the inclusions will need to balanced to allow maximum infusion of the inclusions without significant amounts of excess liquid remaining.

| | |
|---|---|
| 80% | Wine (infusion liquid) |
| 20% | Grape skin or grape seed extract (bioactive |
| | substance) |
| 0.5% | Synthetic or natural flavour |

All ingredients were mixed in a large vessel at room temperature and stirred slowly to ensure that the grape skin/seed extract and flavour was well blended with the wine.

The infusion mixture was then combined with the inclusions (currants) in a suitable vessel and the vessel was rotated to assist with complete mixing (eg by tumbling). The infusion mixture was infused into the inclusions over a period of time (eg 24 hours) with regular turning (mechanically or by hand) to provide optimal flavour, taste and deliverability.

The inclusions were then allowed to stand for 30 minutes before being filtered and strained prior to being placed onto racks for drying. The inclusions were dried in a warm room (37 to 40° C.) with air flowing across the inclusions for 48 hours.

The above process can be repeated to increase the intensity of the flavour and concentration of bioactive substance in the inclusions.

### Example 6

If the target bioactive substance to be administered is not a powder, then instead of dispersing the inclusions into a chocolate block, it is preferable to coat the inclusions with the chocolate. These chocolate ‘drops’ could then be consumed in a similar fashion as tablets—but preferably with an overall more pleasant experience.

Infusion Mixture

The following mixture (25 litres) was sufficient to infuse 125 kg of sun muscats or another suitably sized dried fruit.

| | |
|---|---|
| 70% | Wine (infusion liquid) |
| 30% | Grape skin or grape seed extract (bioactive |
| | substance) |
| 1% | Synthetic or natural flavour |

The flavours were used in this mixture to infuse further characteristic notes and varietal wine flavours into the sun muscats.

All of the above ingredients were added to a large plastic vessel at room temperature. The mixture was stirred slowly with a large spoon, or similar, to ensure that the grape skin extract and flavour was well blended with the wine. The mixture was prepared immediately prior to infusion.

Infusing the Sun Muscats

The sun muscats were evenly split amongst an appropriate number of suitable vessels which could be rotated to assist mixing. Any clumps of sun muscats were broken by hand. The infusion mixture was equally divided amongst the vessels and the vessels rotated 10 times to mix well and there are no clumps of the sun muscats. The vessels were rotated at regular intervals to ensure even infusion of the sun muscats. If the vessels are not rotated regularly, excessive amounts infusion mixture will be left behind and not taken up by the sun muscats. The vessels were left overnight at 18° C. to 25° C.

The next day each vessel was rotated to mix well again and allowed to stand for 30 minutes. The sun muscats were filtered and strained to remove as much surface liquid as possible. Filtration and straining is only necessary if there is an excessive amount of infusion mixture still left in the drum. The sun muscats were then spread evenly onto racks for drying. The racks were placed in a warm room (37-40° C.) with air flowing across the sun muscats for 45 to 48 hours. The sun muscats were well dried, free of surface moisture, free flowing and not excessively ‘sticky’.

Coating Sun Muscats with Chocolate

The base chocolate (prepared as described in example 1 to contain grape seed and or skin extract) is flavoured to correspond to the wine used in the infusion. Addition of the flavours can result in “thickening” of the chocolate. If this occurs, add up to no more than 1.5 kg of PGPR (not more) to each 500 kg batch and mix well to reduce viscosity. If a number of flavours are added, then each should be added individually. The chocolate and flavours mixture was mixed for at least 10 to 15 minutes with a high shear mixer before pumping to the finn coater. The chocolate was kept at 40-45° C. and stirred constantly.

A thin layer of chocolate was sprayed onto the finn coater belt. This provided a surface for the centres to ‘grip’ to, aiding tumbling and preventing the centres from sliding on the belt. Any clumps of sun muscats were broken before transferring to the finn coater.

Coating was carried out to a ratio of 0.5, that is, the amount of chocolate is equivalent to 50% of the total weight of the infused sun muscat. For example, 80 kg of infused sun muscats will utilize 40 kg of chocolate to provide a total weight of 120 kg of finished product.

After coating with chocolate, the ‘wine drops’ were transferred to a conventional pan and can be dusted with cocoa powder (ie to 40 kg of product, 500 grams of cocoa powder was added). The pan was run at a suitable speed for 10 minutes to ensure even coating.

### Example 7

In this example, the dispersion of a bioactive powder in a chocolate product made using standard chocolate manufacturing methods was investigated.

Chocolate Preparation

A 1 kg batch of red wine chocolate was prepared in the laboratory containing:

| | | | |
|---|---|---|---|
| Castor sugar | 400 g | Lecithin | 5 g |
| Cocoa liquor | 350 g | Natural vanilla | 4 g |
| Full cream milk | 140 g | PGPR | 1 g |
| powder | | | |
| Cocoa butter | 100 g | Grape seed | 4.5 g |
| | | powder | |

The cocoa liquor and cocoa butter were melted, added to the conche and then blended together. The castor sugar, full cream milk powder and 50% of the lecithin were added in sequence while the conche was running—ensuring that each ingredient was well blended prior to adding the next ingredient. The mixture was conched until the desired particle size was achieved (5 hours). Then the remaining lecithin, natural vanilla and PGPR were added whilst the conche was running until the ingredients were well combined. The grape seed powder was then added directly to the chocolate mixture while the conche was running. The conche continued until al the ingredients were well blended.

The chocolate was then removed from the conche and moulded into 100 g blocks. One block of chocolate was chosen at random and broken into six equal pieces. Each of these pieces was then assessed for antioxidant capacity using an established oxygen radical absorbance capacity (ORAC) assay.

ORAC Testing Methodology

Six samples were submitted for the ORAC assay. Grape skin is a well known antioxidant. If it was dispersed substantially evenly throughout the chocolate, the ORAC testing should be consistent across the six chocolate batches manufactured using the same methodology.

Samples were ground and approximately 50 mg was solubilised in 5 ml of methanol. The samples were vortexed, sonicated for 30 minutes and centrifuged for 5 minutes (1900 RCF). The supernatant was collected and taken to dryness. Samples were resolubilized in methanol at 10 mg/ml. The samples analysed were as follows:
Sample A—red wine chocolate batch 1
Sample B—red wine chocolate batch 2
Sample C—red wine chocolate batch 3
Sample D—red wine chocolate batch 4
Sample E—red wine chocolate batch 5
Sample F—red wine chocolate batch 6

The ORAC assay employed in this study measured the antioxidant scavenging activity in the test sample, against peroxyl radicals induced by 2,2′-azobis (2-amidinopropane) dihydrocholoride (AAPH) at 37° C. Flourescein was used as the fluorescent probe. Hydrophilic ORAC values determined for the samples.

The samples were assayed using the ORAC procedure in serial dilution (×4) with AWA (acetone:water:acetic acid; 70:29.5:0.5), and in quadriplucate, starting with the concentration relevant to the sample, depending on the approximated antioxidant capacity from an initial screen.

A green tea extract was included as a positive control, and the extract was prepared as per the sample preparation. The green tea methanolic extract was directly solubilised in phosphate buffer (pH 7.4) and assayed as per the other samples, with the exception of the AWA being substituted with phosphate buffer.

Trolox, a water soluble analogue of vitamin E, was used as a reference standard. A trolox standard curve was established from trolox standards prepared at 100, 50.25 and 12.5 μM in AWA.

20 μl samples/standards/control/blank (AWA), 10 μl fluorescein (6.0×10⁻⁷ M) and 170 μl AAPH (20 mM) were added to each well. Immediately after loading, the plate was transferred to the plate reader preset to 37° C., and the fluorescence was measured 35 times at one minute intervals. The fluorescence readings were referenced to solvent blank wells. The final ORAC values were calculated using a regression equation between the trolox concentration and the net area under the fluorescein decay curve, and were expressed as micromole trolox equivalents (TE) per g of sample.

Results and Discussion

TABLE 1
Yield of extract from each sample

| Sample | Sample mass (mg) | Extract mass (mg) | Yield (%) |
|---|---|---|---|
| A | 49.8 | 25.6 | 51 |
| B | 45.0 | 24.1 | 54 |
| C | 44.7 | 23.6 | 53 |
| D | 47.8 | 29.9 | 63 |
| E | 44.4 | 27.6 | 62 |
| F | 46.5 | 29.4 | 63 |
| Green tea | 48.5 | 16.3 | 34 |

TABLE 2
Antioxidant capacity of six sugar/chocolate samples extracted
with methanol, compared to a green tea methanol extract
(values are mean ± standard error of the mean)

| Sample | ORAC value (μmol TE/g of sample) |
|---|---|
| A | 90 ± 6.1 |
| B | 115 ± 10.3 |
| C | 77 ± 3.7 |
| D | 113 ± 11.3 |
| E | 122 ± 9.9 |
| F | 119 ± 8.19 |
| Green tea | 1793 ± 93.5 |

Conclusion

ORAC values ranged from a high of 122 μmol TE/g of sample to a low 77 μmol TE/g of sample when extracted with methanol. The considerable range clearly demonstrates that dispersion of the grape seed powder was uneven and inconsistent when standard chocolate manufacturing methods and technology were used.

The conventional method fails to provide an even distribution so that each pip in a block of chocolate contains an equal dose of the bioactive substance. Without this consistent and even distribution, it is not possible to provide precise doses of the bioactive substance to a subject and consistently provide a therapeutic effect.

### Example 8

This example illustrates an alternative inclusion infusion methodology.

This method utilises vacuum as a means of infusing inclusions, such as currants or sun muscats, with an infusion mixture.
Fill a suitable plastic bag with the correct weight of inclusions. Add to the bag the correct volume of infusion mixture (usually 20%).
Place the bag inside a vacuum packaging machine ensuring that no liquid escapes the bag.
Operate the machine and so that the contents of the bag are sealed under vacuum.
Remove the bag and store under optimum conditions (18 to 22° C.) for 24 hours.
Remove the inclusions from the bags and shake the infused inclusions well in a filter to remove as much excess liquid as possible. Discard the filtrate. Spread the infused inclusions on a drying rack and place in a warm room (40° C.) with air flowing across the currants overnight.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

1. A drug delivery system for a bioactive powder comprising the bioactive powder and a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, wherein the bioactive powder is substantially evenly dispersed throughout the delivery system.

2. The delivery system according to claim 1 wherein the lipid is selected from the group consisting of cocoa butter, a cocoa butter equivalent, milk fat and mixtures thereof.

3. A chocolate product having an even distribution of a bioactive powder comprising the drug delivery system according to claim 1, wherein the bioactive powder is dispersed into the lipid prior to addition to a chocolate mixture.

4. A method for delivering bioactive powders, the method comprising the step of combining the bioactive powder with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the bioactive powder is substantially evenly dispersed throughout the lipid.

5. Use of a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate in the manufacture of a formulation comprising a bioactive powder, wherein the bioactive powder is substantially evenly distributed throughout the formulation.

6. A cocoa product having an even distribution of a bioactive powder, wherein the bioactive powder is substantially evenly dispersed in a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof.

7. A method for manufacturing a chocolate product comprising a bioactive powder, said method comprising:
(a) first, combining the bioactive powder with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the powder is substantially evenly distributed in the lipid, and then
(b) combining the lipid mixture with the other chocolate ingredients until the lipid mixture is substantially evenly dispersed in the chocolate product.

8. A method for improving the bioavailability of a lipophilic bioactive powder, said method comprising the step of combining the bioactive powder with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the bioactive powder is substantially evenly dispersed throughout the lipid.

9. A method for minimising the level of degradation of a bioactive powder, said method comprising the step of administering the bioactive powder in combination with a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, until the bioactive powder is substantially evenly dispersed throughout the lipid.

10. A method for incorporating bioactive substances into cocoa products, said method comprising the steps of:
(a) combining the bioactive substance with an infusion liquid,
(b) infusing the bioactive liquid mixture into inclusions; and then
(c) combining the infused inclusions with the cocoa product.

11. The method according to claim 10 wherein the infusion liquid comprises flavours to mask the flavour of the bioactive substance.

12. The method according to claim 10 wherein the inclusions are selected from the group consisting of dried fruit, freeze dried fruit, dried fruit pastes, fruit purees, extruded fruit particles, puffed grains, baked grain inclusions, processed cereals and cereal fractions, synthetic substitutes and mixtures thereof.

13. The method according to claim 10 wherein step (b) further comprises a drying step after the infusion.

14. A wine chocolate product comprising:
(a) wine flavoured chocolate comprising chocolate, flavours, and polyphenols powders dispersed in a lipid selected from the group consisting of cocoa butter, cocoa butter equivalents, cocoa butter substitutes, cocoa butter replacers, cocoa butter fractions, cocoa butter improvers, other lipids having a sharp melting curve in the range from 30 to 37° C. and which melt in the range from 35 to 37° C., other lipids which are miscible with chocolate and mixtures thereof, wherein the bioactive powder is substantially evenly distributed throughout the wine flavoured chocolate; and
(b) wine flavoured inclusions comprising inclusions which have been infused with a liquid mixture comprising wine, polyphenols and flavours.

15. The wine chocolate product according to claim 14 wherein the wine flavoured inclusions are individually coated with the wine flavoured chocolate.

16. The wine chocolate product according to claim 14 wherein the wine flavoured chocolate is moulded into blocks wherein the pips each have substantially the same polyphenol content within a typical tolerance for a therapeutic product.

17. The wine chocolate product according to claim 14 wherein the wine flavoured chocolate is centred filled with the inclusions and wine flavoured filling moulded into blocks wherein the pips each have about the same polyphenol content.

18. A method of enhancing the absorption of a polyphenol into the blood of a human comprising administering to the human a wine chocolate product according to claim 14.

19. A method of promoting vascular health of a human comprising administering to the human a wine chocolate product according to claim 14.
